(19) United States
(12) Patent Application Publication
(10) Pub. No.: US 2013/0224280 A1
(43) Pub. Date: Aug. 29, 2013

(54) TRANSDERMAL COMPOSITIONS AND METHODS FOR TREATING STRETCH MARKS

(71) Applicant: Rita Toth, Meredith, NH (US)

(72) Inventor: Rita Toth, Meredith, NH (US)

(21) Appl. No.: 13/773,917

(22) Filed: Feb. 22, 2013

Related U.S. Application Data
(60) Provisional application No. 61/634,106, filed on Feb. 23, 2012.

(51) Int. Cl. A61K 45/06; A61K 31/728; A61K 31/4188; A61K 31/075; A61K 38/39; A61K 36/81; A61K 31/045; A61K 31/355; A61K 36/53

(52) U.S. Cl. CPC A61K 45/06; A61K 31/355; A61K 31/728; A61K 31/4188; A61K 36/53; A61K 38/39; A61K 36/81; A61K 31/045; A61K 31/075
USPC 424/443; 424/745; 424/744; 424/746; 424/125

(57) ABSTRACT

A transdermal delivery device for treating or preventing scars, such as stretch marks, comprising a composition comprising vitamin E, hyaluronan, biotin, Rosmarinus officinalis, collagen, and a heat generating ingredient, and methods therefor.

### CROSS-REFERENCE TO RELATED APPLICATIONS

This application claims priority under 35 U.S.C. §119(e) to U.S. Provisional Application No. 61/634,106, filed Feb. 23, 2012, which is incorporated herein by reference in its entirety.

### BACKGROUND

The present invention relates to compositions and methods for treating scars, such as stretch marks. In particular the present invention relates to compositions and methods for treating scars, such as stretch marks, by transdermal administration of at least one therapeutic ingredient to a subject.

Striae distensae lesions, also known as stretch marks, typically occur on a subject's abdomen, breasts, buttocks, hips, and thighs but can occur on any part of the body. Presumably, the lesions arise due to weakening and/or breaking of elastin tissue in the dermal (middle) skin layer. Stretch marks can occur, for example, during pregnancy, rapid growth spurts in adolescence, hormone level disruption, nutritional deficiency, periods of excessive weight change, or periods of excessive muscle growth. Stretch marks have also been associated with topical or prolonged treatment with corticosteroids. In the United States it is estimated that about 80-90% of pregnant women, 70% of adolescent females, and 40% of adolescent males have stretch marks.

Although striae distensae lesions are not regarded as a medical problem, their cosmetic appearance can cause considerable distress. Numerous creams, ointments and other therapies have been used treat stretch marks, but none are measurably effective at minimizing or reducing their appearance. Furthermore, currently available topical, systemic and/or surgical treatments can cause unwanted side effects including, for example, skin thinning, easy bruising, rash, increased susceptibility to infection, allergic reaction, headache, nausea, and vision impairment. Moreover, topical treatments can be messy or difficult to apply and often have limited treatment time and efficacy. Furthermore, topical delivery of a precise amount of therapeutic agent to a subject can be difficult.

A multilayered skin structure is also largely responsible for the inefficacy of topical treatments. A primary function of the skin is to act as a barrier against the egress of endogenous substances (e.g., water) and the ingress of xenobiotic material (e.g., chemicals and drugs). The outermost portion of the barrier, known as the stratum corneum (SC), is comprised of dead cells interdispersed within a lipid rich matrix. The lipophilic SC exhibits selective permeability and allows only relatively lipophilic compounds to diffuse into the lower and middle layers of the skin. Due to this selective permeability, solute transport across the SC is primarily driven by passive diffusion in accordance with Fick's Law, and no active transport processes have been identified. As a result, the SC is a major obstacle to transdermal drug delivery.

### SUMMARY

Embodiments described herein include, among other embodiments, compositions and methods for the transdermal treatment of scars, such as stretch marks.

For example, some embodiments relate to a transdermal delivery device comprising a composition comprising vitamin E, hyaluronan, biotin, Rosmarinus officinalis, and collagen. In some embodiments, the composition further comprises one or more of jojoba, Lavendula augustifolia, cocoa butter, aloe vera, vitamin C, caffeine, taurine, Salvia officinalis, Pelargonium graveolens, Humulus lupulus, panthenol, linalool, theobroma oil, vitamin H, vitamin B7, ascorbic acid, L-ascorbic acid, ascorbyl acid, ascorbyl palmitate, sodium ascorbyl phosphate, retinyl ascorbate, tetrahexyldecyl ascorbate, magnesium ascorbyl phosphate, or caffeine sodium benzoate.

In specific embodiments, the composition comprises about 2 mg/ml to about 40 mg/ml jojoba; about 2 mg/ml to about 40 mg/ml Lavendula augustifolia; about 1 mg/ml to about 50 mg/ml cocoa butter; about 1 mg/ml to about 50 mg/ml vitamin E; about 0.2 mg/ml to about 25 mg/ml hyaluronan; about 0.5 mg/ml to about 50 mg/ml aloe vera; about 0.5 μg/ml to about 200 μg/ml vitamin C; about 50 mg/ml to about 200 mg/ml caffeine; about 0.2 mg/ml to about 40 mg/ml taurine; about 0.1 mg/ml to about 10.5 mg/ml Rosmarinus officinalis; about 0.8 mg/ml to about 10.7 mg/ml Salvia officinalis; about 0.039 mg/ml to about 19 mg/ml Pelargonium graveolens; about 0.4 mg/ml to about 60 mg/ml Humulus lupulus; about 5 mg/ml to about 40 mg/ml panthenol; and about 1 mg/ml to about 100 mg/ml collagen.

In accordance with any of these embodiments, the device may further comprise a heat generating ingredient. In some embodiments, the heat generating ingredient(s) comprise one or more of menthol, vanillyl butyl ether, caspium, red pepper extract, ginger extract, or heat generating pellets.

In specific embodiments, the composition comprises one or more of menthol, vanillyl butyl ether, caspium, or combinations thereof. In any such embodiments, the composition may comprise about 1% to about 36% menthol, and/or about 0.1% to about 2% vanillyl butyl ether and/or about 0.05% to about 1.5% caspium, each based on the total weight of the composition.

In any embodiment comprising heat generating pellets, the heat generating pellets may comprise charcoal, activated carbon, salt, iron, water, cellulose and vermiculite which produce heat upon contact with air.

In accordance with any of the above-described embodiments, the transdermal delivery device may comprise a polymer. In some embodiments, the polymer is vinyl acetate or acrylate.

In accordance with any of the above-described embodiments, the transdermal delivery device may comprise at least one preservative and/or scent. In some embodiments, the preservative is thiosulfate silica, methylparaben, E-218, propylparaben, butylparaben, ethylparaben, or combinations thereof. In some embodiments, the scent is provided by limonene, geraniol, citral, rose oils, citral oils, or some combination thereof.

In some embodiments, one or more optional ingredient(s) can improve miscibility of the therapeutic or other ingredients. In some embodiments, the miscibility improving ingredient(s) is octyldodecanol, stearyl alcohol, oleyl alcohol, or combinations thereof. In some embodiments, octyldodecanol can be present from about 1 mg/ml to about 30 mg/ml, or about 10 mg/ml to about 15 mg/ml.

In accordance with any of the above-described embodiments, the transdermal delivery device may comprise a backing layer; an adhesive layer disposed on the backing layer; and optionally, a release liner. In some embodiments, the adhesive layer comprises the therapeutic ingredient(s). In alternative embodiments, the transdermal delivery device may comprise a backing layer; a reservoir disposed on the backing layer; and optionally, a release liner. In some embodiments, the reservoir comprises the therapeutic ingredient(s). In some embodiments, the transdermal delivery device comprises an adhesive layer, and the reservoir is disposed between the adhesive layer and the backing layer. In some embodiments, the transdermal delivery device comprises a rate-controlling membrane disposed between the reservoir and the adhesive layer.

In accordance with any of the above-described embodiments, the transdermal device may have a surface area of about 1 cm² to about 150 cm².

Also provided are methods of treating or preventing scars, such as stretch marks, comprising applying a transdermal delivery device as described herein to the skin of a subject in need thereof. In some embodiments, the device is applied to a subject's skin for about 0.5 hours to about 12 hours. In some embodiments, the device is applied once or twice per day for about 12 weeks to about 16 weeks.

### BRIEF DESCRIPTION OF THE DRAWINGS

FIG. 1 shows a schematic drawing of a matrix-type transdermal delivery patch.

FIG. 2 shows a schematic drawing of a reservoir-type transdermal delivery patch.

### DETAILED DESCRIPTION

Described herein are compositions and methods for the transdermal treatment of scars, such as stretch marks.

### Definitions

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein “subject” denotes any animal with or without scars, such as stretch marks, including humans. For example, a subject may be suffering from or at risk of developing scars, such as stretch marks.

As used herein, the phrases “therapeutically effective amount” means that therapeutic ingredient dosage in a subject that provides the specific pharmacological response for which the therapeutic ingredient is administered in a subject in need of such treatment. It is emphasized that a therapeutically effective amount of an ingredient will not always be effective in treating the conditions described herein, even though such dosage is deemed to be a therapeutically effective amount by those of skill in the art. For convenience only, exemplary dosages, ingredient delivery amounts, and therapeutically effective amounts are provided below with reference to adult human subjects. Those skilled in the art can adjust such amounts in accordance with standard practices as needed to treat a specific subject.

As used herein, the term “transdermal” refers to delivery, administration or application of a therapeutic ingredient(s) by means of direct contact with skin. Such delivery, administration or application is also known as dermal or percutaneous.

### Therapeutic Ingredients

The present disclosure relates to transdermal delivery devices that comprise a composition comprising at least one therapeutic ingredient. Therapeutic ingredient(s) can be, for example, any ingredient capable of moisturizing skin, rejuvenating cells, treating burns, stimulating cell growth, regenerating cells, soothing skin, metabolizing energy, promoting collagen production, reducing inflammation, strengthening skin, altering skin tone, hydrating skin, firming skin, burning fat, killing microorganisms, conditioning skin, balancing skin elasticity, preventing cellulite, constricting tissues, softening skin, or carrying an optional ingredient. In some embodiments, the therapeutic ingredient(s) can have antioxidant or fragrant properties.

In some embodiments, the composition comprises one or more of vitamin E, hyaluronan, biotin, Rosmarinus officinalis, collagen, jojoba, Lavendula augustifolia, cocoa butter, aloe vera, vitamin C, caffeine, taurine, Salvia officinalis, Pelargonium graveolens, Humulus lupulus, panthenol, octyldodecanol, and combinations thereof. In some embodiments, the composition comprises vitamin E, hyaluronan, biotin, Rosmarinus officinalis, and collagen. In some embodiments, the composition further comprises one or more of jojoba, Lavendula augustifolia, cocoa butter, aloe vera, vitamin C, caffeine, taurine, Salvia officinalis, Pelargonium graveolens, Humulus lupulus, and panthenol. In some embodiments, the transdermal delivery device comprises all of the above-mentioned therapeutic ingredients. In some embodiments, the transdermal delivery device comprises at least vitamin E, hyaluronan, Rosmarinus officinalis, and collagen.

In some embodiments, the transdermal delivery device comprises a therapeutically effective amount of one or more of the therapeutic ingredient(s). Exemplary, non-limiting amounts are provided below.

Jojoba can be present at any therapeutically effective amount, for example about 2 mg/ml to about 40 mg/ml, or about 8 mg/ml to about 20 mg/ml.

Lavendula augustifolia can be present at any therapeutically effective amount, for example about 2 mg/ml to about 40 mg/ml, or about 8 mg/ml to about 20 mg/ml. Additionally or alternatively, some or all of the Lavendula augustifolia can be replaced with any functionally equivalent ingredient, for example linalool.

Cocoa butter can be present at any therapeutically effective amount, for example about 1 mg/ml to about 50 mg/ml, or about 10 mg/ml to about 25 mg/ml. Additionally or alternatively, some or all of the cocoa butter can be substituted with any functionally equivalent ingredient, for example theobroma oil.

Vitamin E can be present at any therapeutically effective amount, for example about 1 mg/ml to about 50 mg/ml, or about 18 mg/ml to about 25 mg/ml.

Hyaluronan can be present at any therapeutically effective amount, for example about 0.2 mg/ml to about 25 mg/ml, or about 0.5 mg/ml to about 10 mg/ml.

Aloe vera can be present at any therapeutically effective amount, for example about 0.5 mg/ml to about 50 mg/ml, or about 5 mg/ml to about 25 mg/ml.

Biotin can be present at any therapeutically effective amount, for example about 0.5 μg/ml to about 200 μg/ml, or about 30 μg/ml to about 100 μg/ml. Additionally or alternatively, some or all of the biotin can be substituted with any functionally equivalent ingredient, for example vitamin H or vitamin B7.

Vitamin C can be present at any therapeutically effective amount, for example about 1% to about 50%, or about 5% to about 20%. Additionally or alternatively, some or all of the vitamin C can be substituted with any functionally equivalent ingredient, for example ascorbic acid, L-ascorbic acid, ascorbyl palmitate, sodium ascorbyl phosphate, retinyl ascorbate, tetrahexyldecyl ascorbate, or magnesium ascorbyl phosphate.

Taurine can be present at any therapeutically effective amount, for example about 0.2 mg/ml to about 40 mg/ml, or about 5 mg/ml to about 10 mg/ml.

Caffeine can be present at any therapeutically effective amount, for example about 50 mg/ml to about 200 mg/ml. Additionally or alternatively, some or all of the caffeine can be substituted with any functionally equivalent ingredient, for example caffeine sodium benzoate.

Rosmarinus officinalis can be present at any therapeutically effective amount, for example about 0.1 mg/ml to about 10.5 mg/ml, or about 0.1 mg/ml to about 1.5 mg/ml.

Salvia officinalis can be present at any therapeutically effective amount, for example about 0.8 mg/ml to about 10.7 mg/ml, or about 0.8 mg/ml to about 1.7 mg/ml.

Pelargonium graveolens can be present at any therapeutically effective amount, for example about 0.039 mg/ml to about 19 mg/ml, or about 0.039 mg/ml to about 10 mg/ml.

Humulus lupulus can be present at any therapeutically effective amount, for example about 0.4 mg/ml to about 60 mg/ml, or about 0.4 mg/ml to about 40 mg/ml.

Panthenol can be present at any therapeutically effective amount, for example about 5 mg/ml to about 30 mg/ml, or about 10 mg/ml to about 20 mg/ml.

Collagen can be present at any therapeutically effective amount, for example about 1 mg/ml to about 100 mg/ml, or about 20 mg/ml to about 80 mg/ml.

In some embodiments, the therapeutic ingredient(s) is adapted for a maximum strength patch. In some embodiments of the maximum strength patch, at least one of the therapeutic ingredient(s) present in the patch are present at the higher end of the ranges disclosed above, or at even higher amounts. For example, in a maximum strength patch, hyaluronan can be present at any pharmaceutically effective amount, for example about 0.2 mg/ml to about 25 mg/ml, or about 5 mg/ml to about 10 mg/ml; biotin can be present at any pharmaceutically effective amount, for example about 0.5 μg/ml to about 200 μg/ml, or about 50 μg/ml to about 100 μg/ml; vitamin C can be present at any pharmaceutically effective amount, for example about 10% to about 50%, or about 10% to about 20%; and panthenol can be present at any pharmaceutically effective amount, for example about 5 mg/ml to about 40 mg/ml, or about 10 mg/ml to about 25 mg/ml.

In some embodiments, the therapeutic ingredient(s) is adapted for a reduced strength patch. In some embodiments of the reduced strength patch, at least one of the therapeutic ingredient(s) present in the patch are present at the lower end of the ranges disclosed above, or at even lower amounts. For example, in a reduced strength patch, hyaluronan can be present at any pharmaceutically effective amount, for example about 0.2 mg/ml to about 25 mg/ml, or about 0.5 mg/ml to about 5 mg/ml; vitamin C can be present at any pharmaceutically effective amount, for example about 5% to about 50%, or about 5% to about 10%; and panthenol can be present at any pharmaceutically effective amount, for example about 5 mg/ml to about 28 mg/ml, or about 10 mg/ml to about 20 mg/ml.

### Heat Generating Ingredients

In some embodiments, the transdermal delivery device comprises at least one heat generating ingredient. In some embodiments, the transdermal delivery device comprises a composition comprising at least one therapeutic ingredient and at least one heat generating ingredient. In some embodiments, the transdermal delivery device comprises at least one heat generating ingredient that is in a different layer than the therapeutic ingredient(s).

The ability to use heat to treat or prevent scars, such as stretch marks, is uncertain in the art. For example, research shows that heat degrades extracellular matrix proteins such as collagen and elastic fibers. See, e.g., J. Y. Seo & J. H. Chung, Thermal aging: A new concept of skin aging, 2 J. Dermatological Sci. Supplement S13 (2006). Nevertheless, the present composition is effective at reducing scars, such as stretch marks, with the use of a heat generating ingredient(s).

While not being bound by theory, heat can increase the diffusion rate of the therapeutic ingredient(s) across the stratum corneum layer of the skin, can increase the permeation of the therapeutic ingredient(s) through the skin, and can increase permeation of the therapeutic ingredient(s) through the epidermal layer of the skin. Additionally, heat can increase the rate of therapeutic ingredients) release from the patch.

In some embodiments, the heat generating ingredient(s) can generate heat over a period of about 1 hour to about 24 hours, or about 0.5 hours to about 15 hours, or about 10 hours to about 12 hours.

In some embodiments, one or more heat generating ingredient(s) are incorporated into the composition that comprises the therapeutic agents, including a reservoir or adhesive. In some embodiments, the heat generating ingredient(s) are incorporated into a separate layer from the composition that comprises the therapeutic ingredient(s). In some embodiments, the heat generating ingredient(s) are incorporated into a closed compartment such that it has no direct contact with the skin when the device is applied to the skin.

In some embodiments, the heat generating ingredient(s) generate heat upon exposure to air. In some embodiments, the heat generating ingredient(s) can be any compound or substance capable of generating heat. For example, heat generating ingredient(s) can be menthol, vanillyl butyl ether, caspium, any functionally equivalent substitute such as red pepper or ginger extract, or any combination thereof.

In embodiments where the heat generating ingredient(s) is present in the composition, the composition may comprise menthol in an amount of about 2% to about 36%, about 2% to about 20%, or about 1% to about 18%, e.g., about 5%, about 10% or about 16%, by weight, based on the total weight of the composition. Additionally or alternatively, the composition may comprise vanillyl butyl ether in an amount of about 0.1% to about 2%, e.g., about 0.3%, about 0.5%, or about 0.8% vanillyl butyl ether can be provided, by weight, based on the total weight of the composition. Additionally or alternatively, caspium, or any functionally equivalent substitute such as red pepper or ginger extract can be present in an amount equivalent to about 0.1% to about 1%, about 0.09% to about 1.5%, or about 0.02% to about 0.8%, e.g., about 0.02%, about 0.05%, or about 0.09% caspium, by weight, based on the total weight of the composition.

Similar amounts of heat generating ingredient(s) can be used where the heat generating ingredient(s) is present in a separate layer from the composition.

In some embodiments, the heat generating ingredient(s) include pellets that produce heat upon contact with air, for example by exothermic oxidation. In some embodiments, the heat generating ingredient(s) include pellets comprising charcoal, activated carbon, salt, iron, water, cellulose and vermiculite.

In some embodiments, the device includes a combination of multiple heat generating ingredient(s).

In some embodiments, the amount of heat generating ingredients is adapted for regular strength, maximum strength, or reduced strength transdermal delivery devices. For example, in reduced strength devices, heat generating ingredient(s) can be present according to the lower exemplary ranges disclosed above, or lower. Maximum strength devices can comprise heat generating ingredient(s) according to the higher exemplary ranges disclosed above, or higher.

### Optional Ingredients

In some embodiments, the device or composition further includes one or more optional ingredients.

For example, in some embodiments, one or more optional ingredient(s) provides the patch or composition with a scent or fragrance.

In some embodiments, limonene is used to provide a citrus or orange scent or fragrance. In some embodiments, limonene is incorporated into the composition. In some embodiments, about 1 mg/ml to about 40 mg/ml, or about 1 mg/ml to about 15 mg/ml limonene can be incorporated into the composition.

In some embodiments, geraniol is used to provide a scent or fragrance that can be rose, peach, raspberry, grapefruit, red apple, plum, lime, orange, lemon, watermelon, pineapple, or blueberry. In some embodiments, geraniol is incorporated into the composition. In some embodiments, about 1 mg/ml to about 40 mg/ml, or about 1 mg/ml to about 15 mg/ml geraniol can be incorporated into the composition. Additionally or alternatively, some or all of the geraniol can be substituted with rose oils.

In some embodiments, citral is used to provide a lemon scent or fragrance. In some embodiments, citral is incorporated into the composition. In some embodiments, about 1 mg/ml to about 40 mg/ml, or about 1 mg/ml to about 15 mg/ml citral can be incorporated into the composition. Additionally or alternatively, some or all of the citral can be substituted with citral oils.

Additionally or alternatively, one or more optional ingredient(s) is a preservative. Any known preservatives can be used. For example, the preservative can be a paraben compound that possesses antibacterial or antifungal properties. In some embodiments, methylparaben is used as a preservative. In some embodiments, methylparaben is incorporated into the composition with the active ingredients. In some embodiments, about 0.75 mg/ml to about 10 mg/ml, or about 0.75 mg/ml to about 2 mg/ml methylparaben can be incorporated into the composition. Additionally or alternatively, some or all of the methylparaben can be substituted with E-218, propylparaben, butylparaben, ethylparaben, or any other functionally equivalent preservative.

In some embodiments, one or more optional ingredient(s) confers heat resistant properties. For example, thiosulfate silicate can be used to reduce, avoid or minimize decomposition or discoloration of the composition or the patch. In some embodiments, thiosulfate silicate is incorporated into the composition with the therapeutic ingredient(s). In some embodiments, about 0.9% to about 2%, e.g., about 0.9% thiosulfate silica is incorporated into the composition.

In some embodiments, one or more optional ingredient(s) can improve miscibility of the therapeutic or other ingredients. In some embodiments, the miscibility improving ingredient(s) can be octyldodecanol. In some embodiments, octyldodecanol can be present from about 1 mg/ml to about 30 mg/ml, or about 10 mg/ml to about 15 mg/ml. Additionally or alternatively, some or all of the octyldodecanol can be substituted with any functionally equivalent material, for example stearyl alcohol or oleyl alcohol.

### Carrier Material

In some embodiments, the composition is provided in a carrier material. A carrier material can be used in any type of patch, for example a reservoir-type patch or a matrix-type patch. In some embodiments, the carrier material is provided in an adhesive layer. In some embodiments, the carrier material is provided in a reservoir layer. In some embodiments, the composition is blended with the carrier material. For example, in some embodiments the composition is blended with a polymer matrix.

In some embodiments, the carrier material comprises at least one type of the following polymer materials: polyacrylates and derivatives thereof, silicone polymers and derivatives thereof, polyisobutylene (PIB) and derivatives thereof, ethylene-vinyl acetate copolymers and derivatives thereof, styrene-block-co-polymers and derivatives thereof, polyacrylic acids and derivatives thereof, polyoxazolines (PDX) and derivatives thereof, poylurethanes and derivatives thereof, polyolefines and derivatives thereof, polyesters and derivatives thereof. For example, the carrier material can comprise dimethicone copolymer acrylates or C10-30 alkyl acrylate cross-polymers.

In some embodiments, the carrier material comprises acrylates or vinyl acetate in an amount ranging from about 0.5% to about 15%, or about 1% to about 5%, by weight, based on the total weight of the composition.

In some embodiments, the composition is applied to the carrier material. For example, in some embodiments the composition is applied to a fabric carrier. In some embodiments, the carrier material comprises a non-woven fabric composed of nylon, polyethylene, polypropylene, rayon, cellulose-rayon, or polyester. In some embodiments, the carrier material comprises a woven fabric made of, for example, gauze or cellulosic materials.

The carrier material may be adhesive, thus constituting a carrier layer for the therapeutic ingredient(s) as well as an adhesive layer for the adherence of the patch to the skin.

### Transdermal Patch

The patch may be sized and shaped for the intended site of application. In some embodiments, the patch has a surface area in the range of about 1 cm² to about 150 cm², or about 3 cm² to about 50 cm², e.g., about 5 cm², about 10 cm², about 20 cm², about 30 cm² or about 40 cm².

In some embodiments, the patch has a regular shape, such as a square, rectangle, circle, or oval shape. In other embodiments, the patch has an irregular shape.

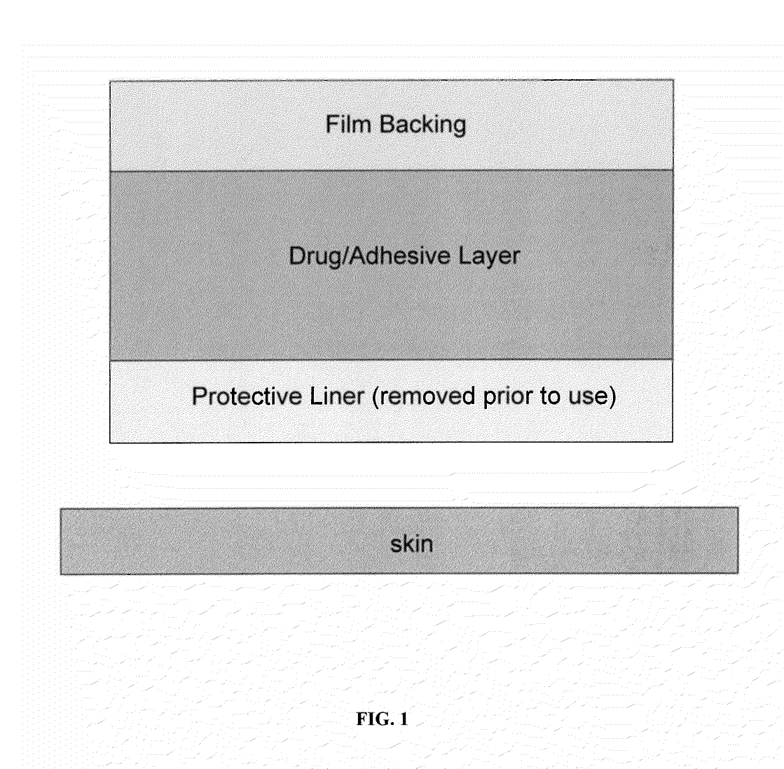

In some embodiments, the transdermal delivery device is a matrix-type patch. Matrix-type patches are known in the art, and any matrix-type patch can be used. For example, some embodiments of the matrix-type patch comprise a backing layer, a drug/adhesive layer, and a release liner (FIG. 1).

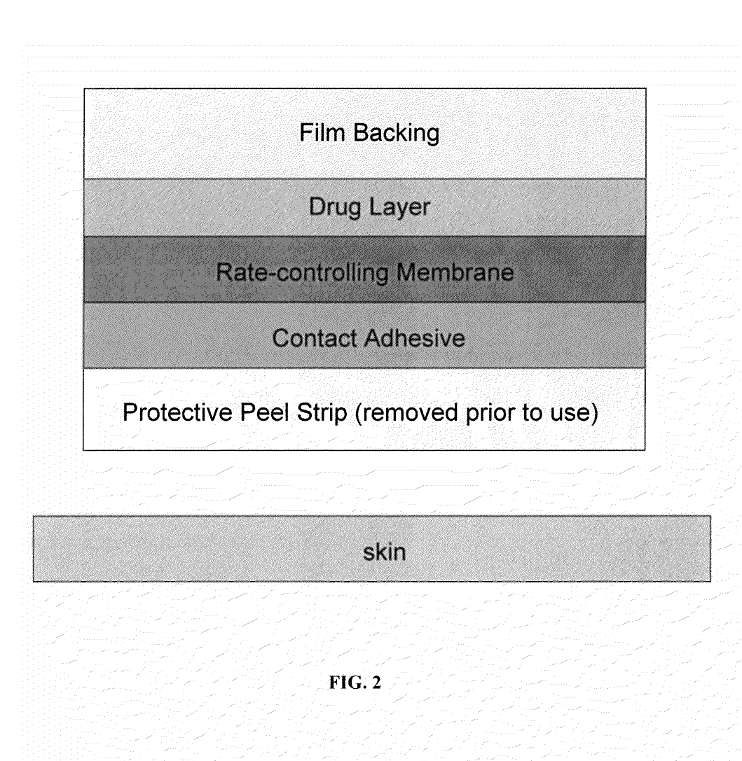

In some embodiments, the transdermal delivery device is a reservoir type patch. Reservoir type patches are known in the art, and any reservoir type patch can be used. For example, in some embodiments the patch can comprise a backing layer, a reservoir disposed on the backing layer, and a release liner. In some embodiments, at least a skin-contacting portion of the reservoir comprises an adhesive. In some embodiments, the surface of the reservoir is provided with an adhesive coating. In some embodiments, the reservoir type patch can comprise a rate-controlling membrane. In some embodiments, the rate-controlling membrane can be disposed between the reservoir and an adhesive layer (FIG. 2).

### Backing Layer

The backing layer may be formed from any material suitable for making transdermal delivery patches, such as a breathable or occlusive material, including fabric or sheet, made of polyvinyl acetate, polyvinylidene chloride, polyethylene, polyurethane, polyester, ethylene vinyl acetate (EVA), polyethylene terephthalate, polybutylene terephthalate, coated paper products, aluminum sheet and the like, or a combination thereof. In some embodiments, the backing layer includes low density polyethylene (LDPE) materials, medium density polyethylene (MDPE) materials or high density polyethylene (HDPE) materials. The backing layer may be a monolithic or a multilaminate layer. In some embodiments, the backing layer is a multilaminate layer including nonlinear LDPE layer/linear LDPE layer/nonlinear LDPE layer. The backing layer can have a thickness of about 0.012 mm (0.5 mil) to 0.125 mm (5 mil); such as about 0.018 mm (0.75 mil) to 0.1 mm (4 mil); or alternatively about 0.025 mm (1 mil) to 0.0875 mm (3.5 mil).

The backing layer can be any color. In some embodiments, the backing layer is a skin-like color. In some embodiments, colors, patterns, shapes, letters, numbers, figures, or characters can be printed on the hacking layer.

### Adhesive

The adhesive, whether provided as part of a reservoir, adhesive coating, or adhesive/drug layer, may be formed from standard pressure sensitive adhesives known in the art. Examples of pressure sensitive adhesives include, but are not limited to, polyacrylates, polysiloxanes, polyisobutylene (PIB), polyisoprene, polybutadiene, styrenic block polymers, and the like. Examples of styrenic block copolymer-based adhesives include, but are not limited to, styrene-isoprene-styrene block copolymer (SIS), styrene-butadiene-styrene copolymer (SBS), styrene-ethylenebutene-styrene copolymers (SEBS), and di-block analogs thereof.

In some embodiments, the adhesive is a carrier material, as described above.

### Release Liner

The transdermal patch may optionally include one or more release liners for storage, handling, or protective purposes. Many suitable release liners are known within the art. The release liner can be removed from the rest of the device by any acceptable means, for example by peeling.

In some embodiments, the release liner can be made of a polymeric material that may be optionally metallized. Examples of suitable polymeric materials include, but are not limited to, polyurethane, polyvinyl acetate, polyvinylidene chloride, polypropylene, polycarbonate, polystyrene, polyethylene, polyethylene terephthalate (PET), polybutylene terephthalate, paper, and combinations thereof. In some embodiments, the release liner can be siliconized. In some embodiments, the release liner can be coated with fluoropolymer. In some embodiments, the release liner includes PET coated with fluoropolymer.

### Reservoir

In embodiments with a reservoir, the reservoir can be a liquid or a gel-type reservoir. Any suitable gelling agent may be used to form an aqueous gel system, for example cellulose materials.

In some embodiments, the reservoir may be formed from any reservoir materials known in the art. For example, the reservoir may be formed from any polymeric material in which the therapeutic ingredient(s) has reasonable solubility and can be delivered within the desired range, such as, a polyurethane, ethylene/vinyl acetate copolymer (EVA), acrylate, styrenic block copolymer, and the like.

As noted above, the reservoir can contain a carrier material.

In some embodiments, the reservoir material can comprise an adhesive to adhere to skin.

The reservoir included in a patch as described herein may optionally contain additional components such as, additives, permeation enhancers, stabilizers, dyes, diluents, plasticizers, tackifying ingredients, pigments, carriers, inert fillers, antioxidants, gelling ingredients, anti-irritants, vasoconstrictors and other materials as are generally known to the transdermal art.

### Rate-Controlling Membrane

Optionally, the patch may contain a rate-controlling membrane disposed between the reservoir and the adhesive layer. The rate-controlling membrane can control the delivery rate of the therapeutic ingredient(s).

In some embodiments, the rate-controlling membrane comprises a polymer. Examples of polymers are known in the art and include, but are not limited to, microporous polyolefin film, acrylonitrile films, polyethylnapthalene, polyethylene terephthalate (PET), polyimide, polyurethane, polyethylene, polypropylene, ethylene-vinyl acetate (EVA), copolymers thereof and mixtures thereof.

In some embodiments, the rate-controlling membrane can also include a heat-sealable material for attaching to other components.

### Method of Treatment

In some embodiments, a method of transdermal administration comprises applying a transdermal delivery device, as described herein, to a subject's skin.

In some embodiments, the method can be used to treat scars. In some embodiments, the method can be used to treat stretch marks. In some embodiments, the method can be used to prevent scars. In some embodiments, the method can be used to prevent stretch marks.

In some embodiments, the device is applied to the skin where a scar, such as a stretch mark, is present or in the process of forming. In some embodiments, the device is applied to the skin where scars, such as stretch marks, commonly occur, for example the abdomen, breasts, buttocks, hips, thighs, or shoulders.

In some embodiments, the device is applied to the skin for a period of time ranging from about 0.5 hours to about 24 hours, or about 1 hour to about 15 hours, or about 10 hours to about 12 hours.

In some embodiments, a device is applied to the skin at least once per day for a total treatment time of at least about 1 week, at least about 4 weeks, at least about 6 weeks, at least about 3 months, or at least about 9 months. In some embodiments, the device is applied to the skin at least twice per day. In some embodiments, the total treatment time is about 12 weeks to about 16 weeks. In some embodiments, the device is applied at least two days per week, at least 3 days per week, at least 4 days per week, at least 5 days per week, or at least 6 days per week for the total treatment time.

### EXAMPLES

### Example 1

A composition for use in a transdermal delivery device is made with the therapeutic ingredients and amounts listed in Table 1 and the optional ingredients and amounts listed in Table 2.

TABLE 1
Therapeutic ingredients

| | Amount of | |
|---|---|---|
| Ingredient | ingredient | |
| Jojoba | 8 | mg |
| Lavendula | 8 | mg |
| Augustifolia | | |
| Cocoa Butter | 10 | mg/ml |
| Vitamin E | 18 | mg/ml |
| Hyaluronan | 0.5 | mg/ml |
| Aloe Vera | 5 | mg/ml |
| Biotin | 30 | μg/ml |
| Vitamin C | 15% | |
| Taurine | 5 | mg/ml |
| Caffeine | 50 | mg/ml |
| Rosmarinus | 0.1 | ml |
| Officinalis | | |
| Salvia Officinalis | 0.8 | mg/ml |
| Pelargonium | 0.039 | mg/ml |
| Graveolens | | |
| Humulus Lupulus | 0.4 | mg/mL |
| Panthenol | 10 | mg/ml |
| Collagen | 20 | mg/ml |

TABLE 2
Optional ingredients

| | Amount of |
|---|---|
| Ingredient | ingredient |
| Limonene | 1 mg/ml |
| Geraniol | 1 mg/ml |
| Citral | 2 mg/ml |

### Example 2

A composition for use in a transdermal delivery device is created based on the composition of Example 1, also with 0.05% caspium.

### Example 3

A composition for use in a transdermal delivery device is created based on the composition of Example 1, also with 10% menthol.

### Example 4

A composition for use in a transdermal delivery device is created based on the composition of Example 1, also with an air activated heat generating ingredient.

### Example 5

A composition for use in a transdermal delivery device is created based on the composition of Example 1, also with 0.5% vanillyl butyl ether.

What is claimed is:

1. A transdermal delivery device comprising:
a composition comprising vitamin E, hyaluronan, biotin, Rosmarinus officinalis, and collagen; and
a heat generating ingredient.

2. The transdermal delivery device of claim 1, wherein the composition further comprises one or more of jojoba, Lavendula augustifolia, cocoa butter, aloe vera, vitamin C, caffeine, taurine, Salvia officinalis, Pelargonium graveolens, Humulus lupulus, panthenol, linalool, theobroma oil, vitamin H, vitamin B7, ascorbic acid, L-ascorbic acid, ascorbyl palmitate, sodium ascorbyl phosphate, retinyl ascorbate, tetrahexyldecyl ascorbate, magnesium ascorbyl phosphate, or caffeine sodium benzoate.

3. The transdermal delivery device of claim 2, wherein the composition comprises: about 2 mg/ml to about 40 mg/ml jojoba; about 2 mg/ml to about 40 mg/ml Lavendula augustifolia; about 1 mg/ml to about 50 mg/ml cocoa butter; about 1 mg/ml to about 50 mg/ml vitamin E; about 0.2 mg/ml to about 25 mg/ml hyaluronan; about 0.5 mg/ml to about 50 mg/ml aloe vera; about 0.5 μg/ml to about 200 μg/ml vitamin C; about 50 mg/ml to about 200 mg/ml caffeine; about 0.2 mg/ml to about 40 mg/ml taurine; about 0.1 mg/ml to about 10.5 mg/ml Rosmarinus officinalis; about 0.8 mg/ml to about 10.7 mg/ml Salvia officinalis; about 0.039 mg/ml to about 19 mg/ml Pelargonium graveolens; about 0.4 mg/ml to about 60 mg/ml Humulus lupulus; about 5 mg/ml to about 40 mg/ml panthenol; and about 1 mg/ml to about 100 mg/ml collagen.

4. The transdermal delivery device of claim 1, wherein the heat generating ingredient comprises one or more of menthol, vanillyl butyl ether, caspium, red pepper extract, ginger extract, or heat generating pellets.

5. The transdermal delivery device of claim 4, comprising one or more of about 1% to about 36% menthol, about 0.1% to about 2% vanillyl butyl ether, or about 0.05% to about 1.5% caspium.

6. The transdermal delivery device of claim 4, wherein the heat generating pellets comprise charcoal, activated carbon, salt, iron, water, cellulose and vermiculite which produce heat upon contact with air.

7. The transdermal delivery device of claim 1, further comprising a polymer.

8. The transdermal delivery device of claim 7, wherein the polymer is vinyl acetate or acrylate.

9. The transdermal delivery device of claim 1, further comprising at least one preservative and/or scent.

10. The transdermal delivery device of claim 9, wherein the preservative is at least one of thiosulfate silica, methylparaben, E-218, propylparaben, butylparaben, or ethylparaben.

11. The transdermal device of claim 9, wherein the scent is provided by at least one of limonene, geraniol, citral, rose oils, or citral oils.

12. The transdermal delivery device of claim 1, further comprising at least one of octyldodecanol, stearyl alcohol, or oleyl alcohol.

13. The transdermal delivery device of claim 1, further comprising:
a backing layer;
an adhesive layer disposed on the backing layer, wherein the adhesive layer comprises the therapeutic ingredient(s); and
optionally, a release liner.

14. The transdermal delivery device of claim 1, further comprising:
a backing layer;
a reservoir disposed on the backing layer, wherein the reservoir comprises the therapeutic ingredient(s); and
optionally, a release liner.

15. The transdermal delivery device of claim 14, further comprising an adhesive layer, wherein the reservoir is disposed between the adhesive layer and the backing layer.

16. The transdermal delivery device of claim 15, further comprising a rate-controlling membrane disposed between the reservoir and the adhesive layer.

17. The transdermal device of claim 1, wherein the device has a surface area of about 1 cm² to about 150 cm².

18. A method of treating or preventing scars or stretch marks comprising applying the transdermal delivery device according to claim 1 to the skin of a subject in need thereof.

19. The method of claim 18, wherein the device is applied to a subject's skin for about 1 hour to about 12 hours.

20. The method of claim 19, wherein the device is applied once or twice per day for about 12 weeks to about 16 weeks.
